(19) United States
(12) Patent Application Publication
(10) Pub. No.: US 2007/0134182 A1
(43) Pub. Date: Jun. 14, 2007

(54) Cosmetic composition comprising an ester of dimerdilinoleic acid and of polyol(s) and a silicone surfactant

(75) Inventors: Momoko Shimizu, Tokyo (JP); Emiko Tokunaga, Kanagawa (JP)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/635,006

(22) Filed: Dec. 7, 2006

Related U.S. Application Data
(60) Provisional application No. 60/754,189, filed on Dec. 28, 2005.

(30) Foreign Application Priority Data
Dec. 8, 2005 (FR) 05 53797

(51) Int. Cl. A61K 8/37

(52) U.S. Cl. 424/64; 424/70.31

(57) ABSTRACT

The present invention relates to an anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa.s, and at least one silicone surfactant.

This non provisional application claims the benefit of French Application No. 05 53797 filed on Dec. 8, 2005 and U.S. Provisional Application No. 60/754,189 filed on Dec. 28, 2005.

The present invention relates to cosmetic compositions for caring for and/or making up the skin and/or the lips, comprising an ester of dimerdilinoleic acid and of polyol(s) and a silicone surfactant.

The compositions according to the invention may show improved gloss and/or colour staying power.

The present invention also relates to a process for making up the human face and/or body using a composition according to the invention.

A composition according to the invention may, for example, be a makeup and/or care product intended to be applied to the body, for example the face, and/or the lips, and may be a lipstick, a lip balm, a lip pencil, a liquid or solid foundation, for example cast as a stick or a dish, a concealer product, a skin colouring product, an eye makeup product, for instance an eyeliner, for example in pencil form, a mascara for example in cake form, or alternatively an eyeshadow or a lip gloss.

Many cosmetic compositions exist for which gloss properties and colour effect properties of the deposited film, after application to the skin and/or the lips, are desired. These properties generally contribute towards the desired aesthetic effect.

Moreover, it is also required that this aesthetic effect be maintained over time, for example in the face of the various external factors liable to affect its gloss and/or colour properties.

In order to give cosmetic compositions good gloss properties and for example good gloss staying power, it has been proposed to use “glossy” oils such as oily polymers, for instance high-viscosity polybutenes, esters of fatty acids or of fatty alcohols with a high carbon number (typically greater than 16), or alternatively certain plant oils.

However, these compounds may under certain circumstances be tacky on application and/or over time, which may result in considerable unpleasant sensations and a feeling of discomfort for the user.

The inventors have been able to observe, surprisingly, that the use of certain esters of dimerdilinoleic acid and of polyol(s), or an ester thereof, in combination with a silicone surfactant, for instance polyglyceryl-3 polymethylsiloxyethyl dimethicone, for preparing a cosmetic composition, makes it possible to give these compositions improved colour staying power without affecting their combination of aesthetic properties, such as the gloss effect and the gloss staying power, while at the same time affording a comfortable sensation on application.

For example, a cosmetic composition according to the invention may have the advantageous property of not causing any tacky sensation after application.

For example, the cosmetic composition according to the invention has a gloss and/or a gloss staying power that is/are not affected, or that is/are even improved.

Thus, according to one of these aspects, one subject of the present invention is an anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa.s, and at least one silicone surfactant.

For the purposes of the invention, the term “anhydrous composition” means a composition containing less than 5% by weight, less than 3% by weight, less than 2% by weight, less than 1% by weight of water relative to the total weight of the composition.

According to another of its aspects, a subject of the present invention is a cosmetic composition for caring for and/or making up the skin and/or the lips, comprising at least one ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15,
   and at least one silicone surfactant.

According to another aspect, a subject of the present invention is a cosmetic composition for caring for and/or making up the skin and/or the lips comprising at least one ester of general formula (II) below: 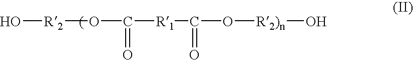
in which:
n is an integer ranging from 1 to 15,
COR′₁CO represents a dimerdilinoleate residue,
OR′₂O represents a diglyceryl residue of general formula (III) below: 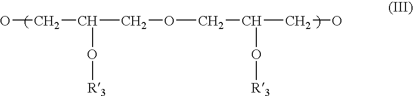
   in which:
   R′₃ represents H or OR′₃ represents a fatty acid residue, and comprising at least one silicone surfactant.

According to another of its aspects, a subject of the present invention is the use of at least one ester of dimerdilinoleic acid and of polyol(s) or an ester thereof, according to the invention, in combination with at least one silicone surfactant, for the preparation of a cosmetic care and/or make-up composition with improved colour staying power.

According to yet another of its aspects, a subject of the present invention is a process for making up and/or caring for the skin and/or the lips, comprising at least one step that consists in applying a composition in accordance with the invention to at least part of a support.

For example, the present invention makes it possible to provide cosmetic compositions whose gloss staying power and/or gloss is not affected, or is even improved, while at the same time showing improved colour staying power.

For instance, the present invention makes it possible to provide compositions whose colour staying power is improved over time, for example with regard to various external attacking factors, such as meals or contact with fabrics.

For example, the present invention makes it possible to provide cosmetic compositions that do not cause any sensation of tack or discomfort when applied.

### Ester of Dimerdilinoleic Acid and of Pollol(s)

In the expression “ester of dimerdilinoleic acid and of polyol(s) or an ester thereof”, the term “or an ester thereof” is intended to denote one of the derivatives of these esters of dimerdilinoleic acid and of polyol(s) obtained either by reaction of alcohol function(s) of the polyol, which are not employed in bonds of ester type with acid functions of the dilinoleic acid, with one or more carboxylic functions of acid molecules other than dilinoleic acid or alternatively by reaction of acid function(s) of the dilinoleic dimer, which are not employed in bonds of ester type with alcohol functions of the polyol, with alcohol functions of alcohol molecules other than the polyol.

### Dimerdilinoleic Acid

The dimerdilinoleic acid that is suitable for use in the present invention may be obtained by polymerization reaction, for example by intermolecular dimerization of at least one linoleic acid.

The oxidation stability of the compound may be improved by hydrogenating the double bonds remaining after the dimerization reaction.

The dimerdilinoleic acid may also be obtained by dimerization of the hydrogenated form of linoleic acid.

The hydrogenated form of the acid or of the diacid may be partial or total, and may correspond, for example, to the saturated form, which is more oxidation-stable.

As indicated previously, the carboxylic functions of the dimerdilinoleic acid residue not engaged in the ester bond with the polyol residue(s) may be engaged in other ester bonds with other alcohol functions of alcohol molecules other than the polyol(s).

These alcohol molecules or residues may be monoalcohols or polyols.

As examples of alcohol residues that are suitable for use in the invention, mention may be made of hydrocarbon-based compounds comprising a hydroxyl function and containing from 4 to 40 carbon atoms, from 6 to 36 carbon atoms, from 8 to 32 carbon atoms, f from 16 to 28 carbon atoms and from 18 to 24 carbon atoms.

As examples of monoalcohols that are suitable for the invention, mention may be made, in a non-limiting manner, of butanol, pentanol, propanol, hexanol, heptanol, octanol, decanol, dodecanol, hexadecanol, octadecanol, eicosadecanol, phytosterol, isostearol, stearol, cetol, behenol, etc.

### Polvols

The term “polyol” is intended to denote any hydrocarbon-based compound comprising at least two hydroxyl functions and containing from 4 to 40 carbon atoms, from 6 to 36 carbon atoms, from 8 to 32 carbon atoms, from 16 to 28 carbon atoms and or from 18 to 24 carbon atoms.

The hydrocarbon-based chains may be interrupted, where appropriate, by the presence of at least one heteroatom, for example an oxygen atom.

A polyol or a polyol ester that is suitable for use in the present invention may comprise, for example, from 2 to 12 hydroxyl functions, from 2 to 8 hydroxyl functions, or from 4 to 6 hydroxyl functions.

Where appropriate, the hydroxyl functions, other than those already employed in an ester bond with the dimerdilinoleic acid, may also be employed, wholly or partly with other ester bonds via reactivity with acid molecules other than the dimerdilinoleic acid.

The polyol or an ester thereof that is suitable for use in the present invention may, for example, be chosen from linear, branched, cyclic or polycyclic, saturated or unsaturated alcohols.

Thus, the polyol may be chosen, for example, from a diol, a triol, a tetraol, or a pentaol, or an ester thereof.

The polyol may be a diol, or an ester thereof, chosen for example from a fatty alcohol dimer, a monoglycerol or polyglycerol, a C₂₋₄ monoalkylene or polyalkylene glycol, 1,4-butanediol and pentaerytiritol.

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, undecanediol, dodecanediol, tridecanediol, tetradecanediol, pentadecanediol, hexadecanediol, nonadecanediol, octadecenediol, cyclohexanediol, diglycerol, erythritol, pentaerythritol, xylitol, sorbitol, ethylene glycol and xylene glycol, and isomers thereof.

A fatty alcohol dimer may also be the product of hydrogenation, for example catalytic hydrogenation, of a fatty acid dimer, which is itself obtained by dimerization of at least one C₈ to C₃₄, C₁₂ to C₂₂, C₁₆ to C₂₀, or C₁₈ unsaturated fatty acid.

A fatty alcohol dimer may range from C₁₆ to C₆₈, for example from C₂₄ to C₄₄, from C₃₂ to C₄₀, or may be of C₃₆.

According to one embodiment, a fatty alcohol dimer may be a diol dimer which may be the product of hydrogenation of dilinoleic diacid. It may be in a saturated form.

A fatty alcohol dimer may be, for example, a dilinoleol dimer.

As an example of a diol that may be suitable for use in the invention, mention may be made of diglycerol.

This compound is a glycerol dimer resulting from the condensation of two molecules of glycerol, with the loss of a water molecule.

The term “diglycerol” denotes any isomer combination that can result from such a condensation, for instance linear isomers, branched isomers and, where appropriate, cyclic isomers resulting from an intramolecular dehydration of a diglycerol molecule.

The diglycerol may be obtained via any process known to those skilled in the art and for example via those described in patent EP 0 750 848.

As examples of acid molecules that can interact with one or more hydroxyl functions of the polyol, not employed in the ester bond with the dimerdilinoleic acid, mention may be made, in a non-limiting manner, of molecules derived from isostearic acid, behenic acid, phytosteric acid, stearic acid or cetylic acid.

An ester that is suitable for use in the present invention may be obtained by reacting a polyol or an ester thereof with a dimerdilinoleic acid, in a molar ratio of about 1.0:0.2-1.0.

An ester that may be suitable for use in the present invention may for example, be obtained by reacting a dimerdilinoleic acid with a dilinoleol and, where appropriate, at least one additional monoalcohol for example chosen from behenol, isostearol, phytosterol, stearol and cetol, and mixtures thereof

Thus, an ester used in the context of the present invention may be used in the form of a mixture of various esters, for example.

An ester that is suitable for the invention may be obtained, for example, by reacting a glycerol, an isostearic acid and a dimerdilinoleic acid, for example, in a molar ratio of 1.0:0.2-1.0:0.5-0.9.

As examples of esters of dimerdilinoleic acid and of polyol(s) or an ester thereof suitable for the invention, mention may be made of the esters described in patent applications JP 2004-256515 and JP 2005-179377.

An ester of dilinoleic acid and of polyol(s) or an ester thereof suitable for use in the present invention may have a molecular weight ranging from about 2000 to about 25 000 g/mol, from about 4000 to about 20 000 g/mol, from about 5000 to about 20 000 g/mol, from about 7000 to about 15 000 g/mol or from about 8000 to about 10 000 g/mol.

According to one embodiment, an ester in accordance with the invention may comprise an alternating sequence of dimerdilinoleate residue(s) and of residue(s) related to the said polyol(s), and for example to the said diol(s), the said polyol(s) or diol(s) being, for example, as defined above.

Thus, in such a configuration, each of the two ends of the said sequence may bear, respectively, a unit OR′ and OR″ with R′ and R″ representing, independently of each other, a hydrogen atom or OR′ and OR″ representing, independently of each other, a C₂ to C₃₆, C₈ to C₂₄, C₁₂ to C₂₀ or C₁₆ to C₁₈ hydrocarbon-based monoalcohol residue.

According to one embodiment, R′ and R″ may both represent a hydrogen atom.

According to one embodiment, OR′ and OR″ may both represent an identical or different hydrocarbon-based monoalcohol residue.

As examples of hydrocarbon-based monoalcohol residues OR′ and OR″ that may be suitable for the invention, mention may be made of fatty alcohol residues.

According to one embodiment, an ester of dimerdilinoleic acid and of polyol(s) or an ester thereof that may be suitable for use in the present invention may have the general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue that may range from C₁₆ to C₆₈, from C₂₄ to C₄₄, from C₃₂ to C₄₀, or may be of C₃₆,
OR₃ represents a monoalcohol residue that may range from 4 to 40 carbon atoms, for example from 6 to 36 carbon atoms, for example from 8 to 32, for example from 16 to 28, or for example from 18 to 24 carbon atoms, and
n is an integer ranging from 1 to 15, from 2 to 10 or from 5 to 7.

According to one embodiment, OR₂O may represent a dimerdilinoleyl residue.

Moreover, OR₃ may represent a hydrocarbon-based monoalcohol residue chosen, for example, from behenyl, isostearyl and phytosteryl residues, and mixtures thereof.

According to another embodiment, the ester of dimerdilinoleic acid and of polyol(s) or an ester thereof that may be suitable for use in the invention may, have the general formula (II) below: 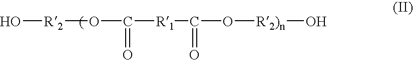
in which:
n is an integer ranging from 1 to 15, from 2 to 10 or from 5 to 7,
OCR′₁CO represents a dimerdilinoleate residue,
OR′₂O represents a diglyceryl residue of general formula (III) below: 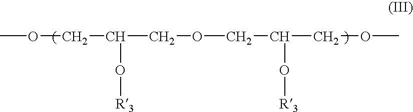
   in which:
   R′₃ represents H or OR′₃ represents a fatty acid residue that may range from C₈ to C₃₄, for example from C₁₂ to C₂₂, for example of C₁₆ to C20 or for example may be of C₁₈.

According to one embodiment, the fatty acid residue represented by OR′₃ may be an isostearyl residue.

The viscosity of an ester of dimerdilinoleic acid and of polyol(s) or an ester thereof, according to the invention, may be measured according to any process known to those skilled in the art, and for example according to the conventional process described hereinbelow.

The viscosity may be measured using a cone/plate or parallel plate viscometer of Ares type (TA-Instrument) operating in kinetic sweep mode over a shear range of about 1-1000 s⁻¹ to induce a flow tension at about 1000 Pa.

The cone/plate or parallel plates may consist of a material selected from the group constituted of stainless steel, acrylic resins or polyphenylene sulfide (PPS resin).

The cone/plate diameter may be 25 mm (cone angle 0.10 radiant).

The measurement is performed at about 25° C.

Before any measurement, the stability of the sample is checked by means of the dynamic sweep period test, which makes it possible to determine if the sample is stable per se.

The shear viscosity is determined using the ETA value in the plateau region according to the flow.

The dynamic sweep period is determined at a frequency of 1.0 Hz over a period of 600 seconds.

The measurements at constant sweep rate are performed with a rate ranging from 1.0 to 1000 s⁻¹ and for example from 1.0 to 100 s⁻¹.

The viscosity of an ester of dimerdilinoleic acid and of polyol or an ester thereof suitable for use in the invention may range from about 20 000 mPa.s to about 150 000 mPa.s, from about 40 000 mPa.s to about 100 000 mPa.s or from about 60 000 mPa.s to about 80 000 mPa.s.

An ester that is suitable for the invention may be chosen for example from the esters having the following INCI nomenclature: polyglyceryl-2 isostearate dimerdilinoleate copolymer, bis-behenyl/isostearyl/phytosteryl dimerdilinoleyl dimerdilinoleate, and mixtures thereof.

Such compounds may be obtained, for example, under the reference Hailuscent ISDA (Kokyu Alcohol) and Plandool-G (Nippon Fine Chemical Company Ltd).

An ester of dimerdilinoleic acid and of polyol(s) or an ester thereof suitable for use in the invention may be present in the cosmetic compositions according to the invention in an amount sufficient to give these compositions improved cosmetic properties, for example in terms of mean gloss staying power.

An ester according to the invention may be present in a composition in accordance with the invention in a content ranging from about 5% to about 90%, from about 15% to about 80% or from about 20% to about 50% by weight relative to the total weight of the composition.

### Surfacants

The term “surfactant” means a compound having at least one hydrophilic portion and at least one hydrophobic portion. Hydrophilic groups and hydrophobic groups are well known to those skilled in the art.

A cosmetic composition in accordance with the present invention comprises at least one silicone surfactant.

For the purposes of the present invention, the term “silicone surfactant” means a surfactant comprising at least one silicon atom and for example Si—O groups.

As examples of silicone surfactants that may be suitable for the invention, mention may be made of silicone surfactants selected from the group constituted of a polyhydroxylated silicone surfactant and an emulsifying silicone resin or elastomer, and mixtures thereof.

A hydroxylated silicone surfactant that may be used in a cosmetic composition according to the present invention is, for example, described in detail in patent application EP 1213 316.

When introduced in sufficient amount, and in combination with an ester of dimerdilinoleic acid and of polyol(s) or an ester thereof as defined above, it is able for example to improve the gloss staying power, or even also the colour staying power, of the cosmetic compositions according to the invention.

For instance, a polyhydroxylated silicone surfactant that may be used in a cosmetic composition according to the present invention may be represented by the general formula (IV) below:
R¹ₐR³_{b}R³_{c}SIO_{(4-a-b-c)/2} (IV)
in which:
a) a, b and c may be such that a ranges from 1 to 2.5; and b and c, independently of each other, range from 0.001 to 1.5,
b) R¹, which may be identical or different, may be selected from the group constituted of:
   C₁ to C₃₀ alkyl radicals, where appropriate substituted with one or more fluorine atoms and amino and/or carboxyl groups,
   aryl and aralkyl radicals, and
   the radicals of general formula (V):
      —C_{d}H_{2d}—O—(C₂H₄O)ₑ(C₃H₆O)_{f}R⁴ (V)
      in which:
   R⁴ may be a C₁ to C₃₀ hydrocarbon-based radical or a radical R⁵—(CO)— with R⁵ being a C₁ to C₃₀ hydrocarbon-based radical, and
   d, e and f may be integers such that d ranges from 0 to 15, and e and f, independently of each other, range from 0 to 50, and
   combinations thereof,
c) R2 may be represented by the general formula (VI) below:
   -Q-O—X (VI)
   in which:
   Q may be a divalent C₂ to C₂₀ hydrocarbon-based radical which may include at least one ether bond and/or at least one ester bond, and
   X may be a polyhydroxylated hydrocarbon-based radical,
d) R³ may be an organosiloxane group of general formula (VII): 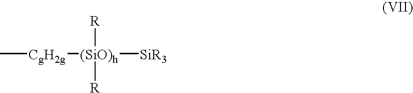
   with:
each of the radicals R possibly representing, independently of each other, a radical selected from the group constituted of C₁ to C₃₀ alkyl radicals, where appropriate substituted with one or more fluorine atoms, and aryl and aralkyl radicals,
g and h possibly being integers such that g ranges from 1 to 5 and h ranges from 0 to 500.

When the radicals R represent a radical selected from the group constituted of C₁ to C₃₀ alkyl radicals, where appropriate substituted with one or more fluorine atoms, aryl radicals and aralkyl radicals, they may have the same meaning as the radical R¹ as defined above.

It should be noted that the radicals R¹, R² and R³ of the silicone polymers of general formula (I), as defined above, may be randomly or statistically distributed, i.e. they may appear in the structure of the polymer without any determined order. Similarly, R¹, R² and R³ may respectively feature radicals of different nature in a compound of general formula (1).

According to one embodiment,

in a):
a may for instance range from 1.2 to 2.3 and, for example, b and c, independently of each another, may range from 0.05 to 1;

in b):
when R¹ is an alkyl radical, it may be a C₁ to C₃₀ alkyl radical, a C₂ to C₂₅ alkyl radical, a C₃ to C₂₀ alkyl radical, a C₄ to C₁₅ alkyl radical, a C₅ to C₁₀ alkyl radical, or a C₆ to C₈ alkyl radical. For example, it may be a methyl, ethyl, n-propyl, isopropyl, n-butyl, isobutyl, tert-butyl, pentyl, hexyl, heptyl, octyl, nonyl, decyl, undecyl or lauryl radical. It may also be a cycloalkyl radical such as a cyclopropyl, a cyclobutyl, a cyclopentyl or a cyclohexyl. It may also be a linear or branched monounsaturated or polyunsaturated alkyl radical. It may also be an alkyl radical substituted with one or more fluorine atoms, such as trifluoropropyl or heptadecafluorodecyl. It may also be an alkyl radical substituted with one or more amino groups, such as 2-aminoethyl, 3-aminopropyl or 3-(2-aminoethyl)aminopropyl. It may also be an alkyl group substituted with one or more carboxyl groups, such as 3-carboxypropyl;
R¹ may also be an aryl or aralkyl radical such as a phenyl radical, a tolyl radical, a benzyl radical or a phenethyl radical;
R¹ may also be an organic group represented by the general formula (V):
   —C_{d}H_{2d}—O—(C₂H₄O)ₑ(C₃H₆O)_{f}R⁴ (V)

According to one particular embodiment, R¹ may be a hydroxylated radical or a radical derived from the addition reaction of a saturated or unsaturated, linear or branched alkenyl ether, in which d=0 and thus of formula:
—O—(C₂H₄O)ₑ(C₃H₆O)_{f}R⁴

In this case, when e and f are equal to zero, then R₁ is an alkoxy group containing from 4 to 30 carbon atoms, for example a C₄ to C₁₀ lower alkoxy radical, such as butoxy or pentoxy, or a C₁₁ to C₃₀ higher alkoxy radical, such as oleoxy or stearoxy, namely, for example, cetyl alcohol, oleyl alcohol and stearyl alcohol, or a radical derived from an acid or from a fatty acid, such as acetic acid, lactic acid, butyric acid, oleic acid, stearic acid or behenic acid.

When e and f are greater than 1, then R¹ is a hydroxyl radical derived from the addition reaction of an alkylene oxide.

When e and f are equal to zero, d may, for example, be equal to 3, 5 or 11. In this case, R¹, depending on the nature of the substituent R⁴, is an allyl ether, pentenyl ether or undecenyl ether radical, or an allyl stearyl ether, pentenyl behenyl ether or undecenyl oleyl ether radical.

When e and f are other than zero, an alkoxy radical and an ester radical are present via a polyoxyalkylene group.

Irrespective of the nature of e and f, d may, for example, be within the range from 3 to 5.

According to one embodiment, the radical R¹ may be any one of the radicals defined above, or a combination of two or more of these radicals.

For example, R¹ may be an alkyl radical selected from the group constituted of a methyl radical and a lauryl radical, and combinations thereof.

Moreover, when R¹ may represent in the same general formula (I) two or more radicals, for example a methyl radical and a lauryl radical, these radicals appear randomly in the structure, and with a frequency that is specific thereto.

For instance, at least 50% of the radicals R¹, at least 70% of the radicals R¹ and 100% of the radicals R¹, may be methyl radicals;

in c):
Q may for example be a divalent hydrocarbon-based radical selected from the group constituted of:

—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁ —, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

For instance, Q may be a divalent radical selected from the group constituted of —(CH₂)₂— and —(CH₂)₃—.
X may for example be a polyhydroxylated hydrocarbon-based radical comprising at least two hydroxyl residues, and for instance a hydrocarbon-based group selected from the group constituted of glyceryl derivatives and saccharide derivatives.

The glycerol residues may be compounds having the following formulae, in which Q has the same meaning as in the general formula (III), and s and t may be integers within the range from 1 to 20, from 1 to 15, from 1 to 10 and from 1 to 5.

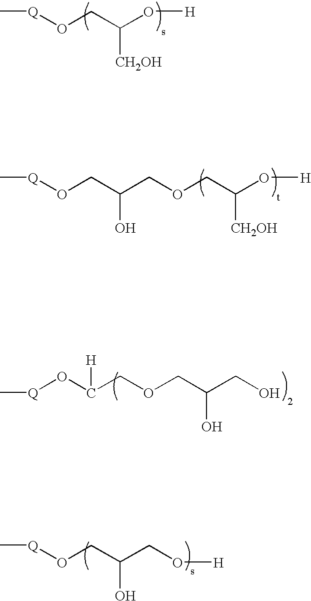

In the above formulae, one or more hydroxyl groups may be replaced with alkoxy groups or ester groups.

The saccharide radicals that may be used in the general formula (III) may be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or of polysaccharide type, such as cellulose or starch.

For example, the saccharide groups may be of monosaccharide or oligosaccharide type;

in d):
each of the radicals R may for example represent, independently of each other, a radical selected from the group constituted of C₁ to C₂₀, C₁ to C₁₀ or C₁ to C₆ alkyl radicals, where appropriate substituted with one or more fluorine atoms. When the radicals R represent a radical selected from the group constituted of the alkyl radicals as defined above, where appropriate substituted with one or more fluorine atoms, they have the same meaning as the radical R¹ as defined above.
g, according to one embodiment, may be equal to 2.
h, according to one embodiment, may be within the range from 1 to 50.

According to one embodiment, the polyhydroxylated silicone surfactant of general formula (IV) that is suitable for use in the present invention may be such that:
a may range from 1 to 1.4, and b and c, independently of each other, range from 0.02 to 0.04, and
R¹ may be a C₁ to C₁₀, C₁ to C₆ or C₁ to C₄, alkyl radical,
R² may be represented by the formula (VIA):
   C₃H₆O[CH₂CH(OH)CH₂O]ₙH (VIA)
   in which n may range from 1 to 5, and
R³ may be represented by the formula (VIIA):
   —C₂H₄(CH₃)₂SiO[(CH₃)₂SiO]ₘSi(CH₃)₃ (VIIA)
   in which m may range from 3 to 9.

According to another embodiment, the polyhydroxylated silicone surfactant of general formula (I), which may be used in the cosmetic compositions according to the invention, is such that:
a may range from 1 to 1.4, and b and c, independently of each other, range from 0.02 to 0.04,
R¹ may be a methyl radical,
R² may be represented by the formula (VIA) in which n may range from 1 to 5, and
R³ may be represented by the formula (VIIA) in which m may range from 3 to 9.

For example, the polyhydroxylated silicone surfactant of general formula (I) used in the cosmetic composition in accordance with the invention may be selected from the group constituted of polyglyceryl-3 polymethylsiloxyethyl dimethicone, laurylpolyglyceryl-3 polymethylsiloxyethyl dimethicone and polyglyceryl-3 disiloxane dimethicone, the respective formulae of which are:
Polyglyceryl-3 polymethylsiloxyethyl dimethicone (formula (VIII)): 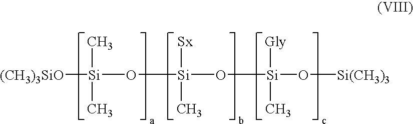
   in which:
Sx: —C₂H₄[(CH₃)₂SiO]ₘSi(CH₃)₃
Gly: —C₃H₆O[CH₂—CH(OH)CH₂O]ₙH
and a=1-1.4, b=0.02-0.04, c=0.02-0.04, m=3-9, n=1-5
   Laurylpolyglyceryl-3 polymethylsiloxyethyl dimethicone (formula (IX)): 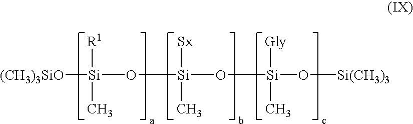
      in which Sx, Gly, a, b, c, m and n have the same meaning as above and R¹ is either a methyl radical or a lauryl radical;
   Polyglyceryl-3 disiloxane dimethicone (formula (X)): 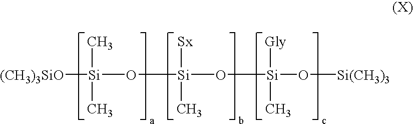
      in which Gly, a, b, c, m and n have the same meaning as above, and
Sx: —O(CH₃)₂SiO—Si(CH₃)₃,
or mixtures thereof.

According to one embodiment, the polyhydroxylated silicone surfactant of general formula (I) may for example be selected from the group constituted of the polymers sold by the company Shin-Etsu under the references KF 6100®, KF 6104® and KF 6105®.

According to yet another embodiment, the polymer sold under the reference KF 6104® may be for instance suitable for preparing the cosmetic compositions in accordance with the invention.

Among the other silicone surfactants that may be suitable for use in the invention, mention may be made, in a non-limiting manner, of the following surfactants:
dimethicone copolyol, such as the product sold under the name Q2-5220 by the company Dow Corning;
dimethicone copolyol benzoate (Finsolv SLB 101 and 201 from the company Finetex),
the cyclomethicone/dimethicone copolyol mixture sold under the name Q2-3225C by the company Dow Corning.

Among the silicone surfactants that may be used in the cosmetic compositions in accordance with the present invention, mention may also be made of the emulsifying silicone resins or elastomers sold by the company Shin-Etsu under the references KSG-310, KSG-320, KSG-330 and KSG-340.

The term “the emulsifying silicone elastomer” means a silicone elastomer comprising at least one hydrophilic chain other than a polyglycerol chain.

For example, the additional emulsifying silicone elastomer may be selected from the group constituted of polyoxyalkylenated silicone elastomers and mixtures thereof.

The polyoxyalkylenated silicone elastomer may be a crosslinked organopolysiloxane that may be obtained via the crosslinking addition reaction of a diorganopolysiloxane containing at least one hydrogen bonded to silicon with a polyoxyalkylene containing at least two ethylenically unsaturated groups.

Polyoxyalkylenated elastomers that may be suitable for use in the invention are for instance described in patents U.S. Pat. No. 5,236,986, U.S. Pat. No. 5,412,004, U.S. Pat. No. 5,837,793 and U.S. Pat. No. 5,811,487.

Polyoxyalkylenated silicone elastomers that may be used include those sold under the names KSG-21, KSG-20, KSG-30, KSG-31, KSG-32, KSG-33, KSG-210, KSG-310, KSG-320, KSG-330, KSG-340 and X-226146 by the company Shin-Etsu, and DC9010 and DC9011 by the company Dow Corning.

Among the silicone surfactants that may be used in the cosmetic compositions in accordance with the present invention, mention may also be made for example of hydrophilic organopolysiloxanes belonging to the dimethicone-polyethylene glycol family and may be chosen for instance from the group comprising dimethicone copolyols, for example cetyldimethicone copolyol, and derivatives thereof.

The hydrophilic organopolysiloxane may be the product sold under the brand name Abil WE09 or Abil EM90 by the company Degussa-Goldschmidt. The hydrophilic organopolysiloxane may also be the product sold under the reference KF-6017 by the company Shin-Etsu.

The organopolysiloxane compound may be totally or partially fluorinated. For example, the lower dialkyl siloxy groups may be substituted with one or more fluorine atoms.

According to one embodiment, a silicone surfactant that may be used in a cosmetic composition in accordance with the present invention may be a silicone surfactant for example chosen from a polyhydroxylated silicone surfactant, dimethicone copolyol, dimethicone copolyol benzoate, dimethicone copolyol phosphates, polyoxyalkylenated silicone elastomers and the cyclomethicone/dimethicone mixture, and mixtures thereof.

The silicone surfactant may be present in the cosmetic compositions in accordance with the present invention in a proportion of from 1% to 30% by weight, from 2% to 25% by weight, from 4% to 20% by weight, from 5% to 20% by weight or from 10% to 15% by weight, relative to the total weight of the composition.

A cosmetic composition according to the invention may also comprise at least one hydrocarbon-based surfactant.

For the purposes of the present invention, the term “hydrocarbon-based surfactant” means a surfactant essentially formed from carbon and hydrogen atoms, and possibly oxygen and/or nitrogen atoms, and free of silicon and fluorine atoms. These agents may, where appropriate, comprise ester, ether, amine and/or amide groups.

The hydrocarbon-based surfactants may be chosen for example from nonionic, anionic, cationic and amphoteric surfactants or alternatively emulsifying surfactants.

Reference may be made to Kirk-Othmer's “Encyclopaedia of Chemical Technology”, volume 22, pp. 333-432, 3rd edition, 1979, Wiley, for the definition of the properties and functions (emulsifying) of surfactants, in particular pp. 347-377 of this reference, for the anionic, amphoteric and nonionic surfactants.

As non-limiting illustrations of nonionic surfactants that may be used in the compositions according to the invention, mention may be made for example of:
oxyethylenated and/or oxypropylenated ethers of glycerol or of fatty alcohols;
fatty acid esters of polyethylene glycol;
oxyethylenated and/or oxypropylenated fatty acid esters of glycerol ethers;
oxyethylenated and/or oxypropylenated fatty acid esters of sorbitol ethers;
copolymers of ethylene oxide and of propylene oxide, condensates of ethylene oxide and propylene oxide with fatty alcohols, polyethoxylated amines or fatty amines, polyglycerolated fatty amides, polyglycerolated diglycolamides, optionally oxyethylenated fatty acid esters of sorbitan, fatty acid esters of sucrose, polyoxyalkylenated fatty acid esters, optionally oxyalkylenated alkylpolyglycosides, alkylglucoside esters, N-alkylglucamine and N-acylmethylglucamine derivatives and amine oxides,
copolymers of propylene oxide and of ethylene oxide;
saccharide esters and ethers;
fatty acid esters of polyols;
and mixtures thereof.

Surfactants that may also be suitable for use in the invention are those of hydrocarbon-based type having solubility parameters δd and δa, according to the Hansen solubility space, which satisfy the following conditions:
16.2≦δ_{d}≦20 (J/cm³)^{1/2}, 16.3≦δ_{d}19 (J/cm³)^{1/2} or 16.9≦δ_{d}≦18 (J/cm³)^{1/2}, and
9.1≦δₐ≦20 (J/cm³)^{1/2}, 10≦δₐ18.1 (J/cm³)^{1/2} or 13≦δₐ≦14.5 (J/cm³)^{1/2}.

The definition of the Hansen solubility parameters is well known to those skilled in the art and is for example described in the article by C.M. Hansen: “The three-dimensional solubility parameters”, J. Paint Technol. 39, 105 (1967). These parameters are also described in document JP-A-08-109121 by Kao and the document by D. W. Van Krevelen “Properties of Polymers” (1990), p. 190.

A hydrocarbon-based surfactant may be selected from the group constituted of partial esters of polyglycerol and of isostearic acid, partial esters of polyglycerol and of oleic acid, and partial esters of sorbitan and of oleic acid, and mixtures thereof.

As hydrocarbon-based surfactants that may be used in the composition according to the invention, it is also possible to select polyglyceryl-2 monoisostearate such as Salacos 41 manufactured or sold by the company Nisshin Oil Mills, polyglyceryl-3 diisostearate such as Lameform TGI manufactured or sold by the company Cognis, polyglyceryl-2 monooleate such as Rylo PG 29 manufactured or sold by the company Danisco Ingredients, and sorbitan monooleate such as Span 80 manufactured or sold by the company Uniqema, and mixtures thereof

The ionic surfactants used in the context of the present invention may be anionic, cationic or amphoteric.

As illustrations of anionic surfactants that are suitable for the invention, mention may be made for example of:
salts of C₁₆-C₃₀ fatty acids, for example those derived from amines, for instance triethanolamine stearate;
polyoxyethylenated fatty acid salts, for example those derived from amines or alkali metal salts, and mixtures thereof;
phosphoric esters and salts thereof such as DEA O1leth-10 phosphate (Crodafos N 10N from the company Croda);
sulfosuccinates such as disodium PEG-5 citrate lauryl sulfosuccinate and disodium ricinoleamido MEA sulfosuccinate;
alkylamido ether sulfates, monoglyceride sulfates and alkyl ether sulfates such as sodium lauryl ether sulfate;
alkylglyceryl sulfonates, alkylsulfonates, alkylamide sulfonates, alkylaryl sulfonates, α-olefin sulfonates and paraffin sulfonates;
isethionates;
acylglutamates such as disodium hydrogenated tallow glutamate (Amisoft HS-21 R sold by the company Ajinomoto), and mixtures thereof.

Triethanolamine stearate is for example suitable for the invention. This product is generally obtained by simple mixing of stearic acid and triethanolamine.

Illustrations of cationic surfactants that may for example be mentioned include:
alkyl-imidazolidiniums such as isostearylethylimidonium ethosulfate,
ammonium salts such as N,N,N-trimethyl-1-docosanaminium chloride (behentrimonium chloride),
optionally polyoxyalkylenated and/or quaternized fatty amine salts, optionally polyoxyalkylenated and/or quaternized fatty acid esters of amino alcohols, quaternary ammonium salts such as tetraalkylammonium, alkylamidoalkyltrialkylammonium, trialkylbenzylammonium, trialkylhydroxyalkylammonium, dialkylamidoalkyldimethyl-ammonium or alkylpyridinium chlorides or bromides and imidazoline derivatives.

A composition according to the invention may also contain one or more amphoteric surfactants, for instance N-acylamino acids such as disodium N-alkylaminoacetates and cocoamphodiacetate and amine oxides such as stearamine oxide, or alternatively silicone surfactants, for instance dimethicone copolyol phosphates such as the roduct sold under the name Pecosil PS 100 by the company Phoenix Chemical, and also ulfobetaines, alkylaminoalkylbetaines, alkylamidoalkylsulfotaines and imidazolium erivatives such as those of amphocarboxyglycinate or of amphocarboxypropionate.

### Physiologically Acceptable Medium

A composition in accordance with the invention comprises a physiologically acceptable medium.

The term “physiologically acceptable medium” is intended to denote a medium that is particularly suitable for the application of a composition according to the invention to the skin and/or the lips. The physiologically acceptable medium is generally suited to the nature of the support onto which the composition is to be applied, and to the aspect in which the composition is intended to be conditioned.

The physiologically acceptable medium may comprise an aqueous and/or water-soluble phase and/or a fatty phase.

According to one embodiment, the aqueous phase or the fatty phase may form the continuous phase of the composition.

This aqueous phase may, where appropriate, be thickened, gelled or structured by also incorporating therein a conventional aqueous gelling agent for example of mineral origin, for instance clay, and/or of organic origin, for instance an aqueous gelling polymer.

According to another embodiment, a composition in accordance with the invention may be in the form of a suspension, a dispersion, a solution, a gel or an emulsion, for example an oil-in-water (O/W), water-in-oil (W/O) or multiple (W/O/W, polyol/O/W or O/W/O) emulsion, or in the form of a cream, a stick, a paste, a foam, a dispersion of vesicles, for instance of ionic or nonionic lipids, a two-phase or multi-phase lotion or a powder.

For the purposes of the present invention, the emulsions contain a lipophilic phase and a hydrophilic phase, the latter phase not systematically being water.

Thus, the cosmetic compositions in accordance with the invention may be in the form of an anhydrous emulsion.

For instance, the composition may contain, for example, a continuous fatty phase, which may contain less than 10% by weight of water, less than 5% by weight of water or less than 1% by weight of water relative to the total weight of the composition.

For example, a cosmetic composition according to the invention may be anhydrous, i.e. may contain less than 5%, less than 3%, less than 2% or less than 1% by weight of water relative to the total weight of the composition. These compositions may then for example be in the form of oily gels, oily liquids, pastes or sticks or alternatively in the form of a vesicular dispersion containing ionic and/or nonionic liquids.

### Fatty Phase

A cosmetic composition in accordance with the present invention may comprise a fatty phase chosen for example from a liquid fatty phase, for instance oils, and a fatty substance that is solid at room temperature (20-25° C.) and atmospheric pressure, and mixtures thereof.

The term “oil” means any fatty substance that is in liquid form at room temperature (20-25° C.) and at atmospheric pressure. The liquid fatty phase may also contain, in addition to oils, other compounds dissolved in the oils, such as gelling agents and/or structuring agents.

The oil(s) may be present in a proportion of from 0.1% to 99% by weight, from at least 1% to 90% by weight, from 5% to 70% by weight, from 10% to 60% by weight or from 20% to 50% by weight relative to the total weight of the cosmetic composition according to the invention.

The liquid fatty phase that may be suitable for preparing a cosmetic composition according to the invention may be selected from the group constituted of volatile or non-volatile, silicone or non-silicone oils, and mixtures thereof.

For the purposes of the present invention, the term “silicone oil” means an oil comprising at least one silicon atom, and at least one Si—O group.

The term “hydrocarbon-based oil” means an oil mainly containing hydrogen and carbon atoms, and possibly oxygen, nitrogen, sulfur and/or phosphorus atoms.

### Volatile Oils

A composition in accordance with the invention may comprise at least one volatile oil.

For the purposes of the present invention, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, for example having a non-zero vapour pressure, at room temperature and atmospheric pressure, for instance having a vapour pressure ranging from 0.13 Pa to 40 000 Pa (10⁻³ to 300 mmHg), ranging from 1.3 Pa to 13 000 Pa (0.01 to 100 mmHg) and ranging from 1.3 Pa to 1300 Pa (0.01 to 10 mmHg).

The volatile hydrocarbon-based oils may be selected from the group constituted of hydrocarbon-based oils containing from 8 to 16 carbon atoms, and for example branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile oils that may also be used include volatile silicones, for instance volatile linear or cyclic silicone oils, for example those with a viscosity ≦8 centistokes (8×10⁻⁶ m²/s), and for instance containing from 2 to 10 silicon atoms or from 2 to 7 silicon atoms, these silicones optionally comprising alkyl or alkoxy groups containing from 1 to 10 carbon atoms. As a volatile silicone oil that may be used in the invention, mention may be made for example of dimethicones with a viscosity of 5 and 6 cSt, octamethylcyclotetrasiloxane, decamethylcyclopentasiloxane, dodecamethylcyclo-hexasiloxane, heptamethylhexyltrisiloxane, heptamethyloctyltrisiloxane, hexamethyldi-siloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

It is also possible to use a mixture of the oils mentioned above.

A cosmetic composition according to the invention may comprise at least one volatile oil in a content ranging from about 2% to about 70% by weight, ranging from about 5% to about 60% by weight or ranging from about 10% to about 50% by weight of volatile oil relative to the total weight of the composition.

### Non-volatile Oils

For the purposes of the present invention, the term “non-volatile oil” means an oil with a vapour pressure of less than 0.13 Pa and for example oils of high molar mass. The non-volatile oils may be hydrocarbon-based oils for instance of animal or plant origin, synthetic oils, silicone oils or fluoro oils, or mixtures thereof.

A cosmetic composition according to the present invention may also comprise at least one non-volatile oil.

The non-volatile oils may be chosen for example from non-volatile hydrocarbon-based oils, which are optionally fluorinated, and/or non-volatile silicone oils.

Non-volatile hydrocarbon-based oils also suitable for the embodiment of the invention that may for instance be mentioned include:
hydrocarbon-based oils of animal origin,
hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C₄ to C₂₄, these chains possibly being linear or branched, and saturated or unsaturated; these oils are for instance heptanoic or octanoic triglycerides, wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818® by the company Dynamit Nobel,
hydrocarbon-based oils of mineral or synthetic origin, for instance:
   synthetic ethers containing from 10 to 40 carbon atoms;
   linear or branched hydrocarbons of mineral or synthetic origin such as petroleum jelly, polydecenes, hydrogenated polyisobutene such as Parleam, and squalane, and mixtures thereof, and for example hydrogenated polyisobutene,
   synthetic esters, for instance oils of formula R₁COOR₂ in which R₁ represents a linear or branched fatty acid residue containing from 1 to 40 carbon atoms and R₂ represents a hydrocarbon-based chain that is for instance branched, containing from 1 to 40 carbon atoms provided that R₁+R₂≧10.

The esters may be chosen for example from fatty acid esters, for instance:
cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythritol tetrahydroxystearate/tetraisostearate;
esters of diol dimers and of diacid dimers, such as Lusplan DD-DA5® and Lusplan DD-DA7®, sold by the company Nippon Fine Chemical and described in patent application FR 03/02809;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
higher fatty acids such as oleic acid, linoleic acid or linolenic acid, and mixtures thereof; and
dialkyl carbonates, the two alkyl chains possibly being identical or different, such as dicaprylyl carbonate sold under the name Cetiol CC® by Cognis;
non-volatile silicone oils, for instance non-volatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyldiphenyl-trisiloxanes and 2-phenylethyl trimethylsiloxysilicates, and dimethicones or phenyltrimethicones with a viscosity of less than or equal to 100 Cst, and mixtures thereof;
and mixtures thereof.

The non-volatile oils may be present in a composition according to the invention in a content ranging from 5% to 90% by weight, from 25% to 80% by weight or from 40% to 70% by weight relative to the total weight of the composition.

### Solid Fatty Substances

The composition according to the invention may also comprise at least one solid fatty phase selected from the group constituted of waxes and pasty fatty substances, and mixtures thereof.

The wax is solid at room temperature (25° C.), with a reversible solid/liquid change of state, having a melting point of greater than 30° C., which may be up to 200° C., a hardness of greater than 0.5 MPa and having in the solid state an anisotropic crystal organization.

It may be a hydrocarbon-based wax, a fluoro wax and/or a silicone wax and may be of animal, plant, mineral or synthetic origin.

It may be chosen, for example, from beeswax, carnauba wax, candelilla wax, paraffin waxes, hydrogenated castor oil, synthetic waxes, for instance polyethylene waxes (preferably with a molecular weight of between 400 and 600) or Fischer-Tropsch waxes, silicone waxes, for instance alkyl or alkoxy dimethicones containing from 16 to 45 carbon atoms, ceresins or ozokerites, for instance isoparaffins with a melting point of less than 40° C., such as EMW-0003 sold by the company Nippon Seirou, α-olefin oligomers, such as the polymers Performa V® 825, 103 and 260 sold by the company New Phase Technologies; ethylene-propylene copolymers, such as Performalene® EP 700, and microcrystalline waxes with a melting point of greater than 85° C., such as Hi-Mic® 1070, 1080, 1090 and 3080, sold by Nippon Seirou, and mixtures thereof

According to one particular embodiment, the wax(es) used in the cosmetic compositions in accordance with the present invention may be present in a content ranging from about 5% to about 30%, from about 5% to about 25%, from about 10% to about 20% or from about 10% to about 15% by weight relative to the total weight of the composition.

A cosmetic composition in accordance with the present invention may also comprise at least one pasty compound.

For the purposes of the present invention, the term “pasty compound” means a fatty compound with a reversible solid/liquid change of state, and comprising at a temperature of 23° C. a liquid fraction and a solid fraction. The term “pasty substance” also means polyvinyl laurate.

For the purposes of the invention, a pasty compound may for example have a hardness at 20° C. ranging from 0.001 to 0.5 MPa and for instance from 0.002 to 0.4 MPa.

Among the pasty compounds that may be used in the composition according to the invention, mention may be made of lanolins and lanolin derivatives, for instance acetylated lanolins, oxypropylenated lanolins or isopropyl lanolate, and mixtures thereof. Esters of fatty acids or of fatty alcohols may also be used, such as those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be used include hydrogenated castor oil derivatives, such as Thixinr® from Rheox.

Mention may also be made of polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) sold by the Japanese company Kokyu Alcohol Kogyo.

As pasty compounds that are for example suitable for formulating the cosmetic compositions in accordance with the present invention, mention may be made of hydrogenated cocoglycerides.

Mention may also be made of pasty silicone compounds such as high molecular weight polydimethylsiloxanes (PDMS) and for instance those containing pendent chains of the alkyl or alkoxy type containing from 8 to 24 carbon atoms, and having a melting point of 20-55° C., for instance stearyl dimethicones, such as those sold by the company Dow Corning under the trade names DC2503® and DC25514®, and mixtures thereof.

### Dyestuff

A cosmetic composition according to the invention may also comprise at least one dyestuff.

Such a dyestuff may be chosen, for example, from water-soluble or water-insoluble, liposoluble or non-liposoluble, organic or mineral dyestuffs, for instance of the type such as pigments or nacres, conventionally used in cosmetic compositions.

The term “pigments” should be understood as meaning white or coloured, mineral or organic particles that are insoluble in an aqueous solution, which are intended to colour and/or opacify the resulting film.

The pigments may be present in a proportion of from 0.5% to 30% by weight, from 5% to 25% by weight or from 10% to 20% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC BALL® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The term “nacres” should be understood as meaning coloured particles of any form, which may or may not be iridescent, for example produced by certain molluscs in their shell, or alternatively synthesized, and which have a colour effect via optical interference.

The nacres may be selected from the group constituted of nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica®, Flamenco® and Duochrome® (on mica base) sold by the company Engelhard, the Timiron® nacres sold by the company Merck, the Prestige® nacres on mica base sold by the company Eckart and the Sunshine® nacres on synthetic mica base sold by the company Sun Chemical.

The nacres may for example have a yellow, pink, red, bronze, orange, brown and/or coppery colour or glint.

As illustrations of nacres that may be used in the context of the present invention, mention may be made of gold-coloured nacres sold for instance by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold for example by the company Merck under the names Bronze fine (17384) (Colorona) and Bronze (17353) (Colorona) and by the company Engelhard under the name Super bronze (Cloisonne); the orange nacres sold for isntance by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold for example by the company Engelhard under the names Nu-antique copper 340XB (Cloisonne) and Brown CL4509 (Chromalite); the nacres with a copper glint sold for isntance by the company Engelhard under the name Copper 340A (Timica); the nacres with a red glint sold for example by the company Merck under the name Sienna fine (17386) (Colorona); the nacres with a yellow glint sold for instance by the company Engelhard under the name Yellow (4502) (Chromalite); the red-tinted nacres with a golden glint sold for example by the company Engelhard under the name Sunstone G012 (Gemtone); the pink nacres sold for instance by the company Engelhard under the name Tan opale G005 (Gemtone); the black nacres with a golden glint sold for example by the company Engelhard under the name Nu antique bronze 240 AB (Timica); the blue nacres sold for instance by the company Merck under the name Matte blue (17433) (Microna); the white nacres with a silvery glint sold for example by the company Merck under the name Xirona Silver; and the golden-green pinkish-orange nacres sold for instance by the company Merck under the name Indian summer (Xirona), and mixtures thereof.

The cosmetic composition according to the invention may also comprise at least one water-soluble or liposoluble dye in a content ranging from 0.5% to 30% by weight and for example ranging from 5% to 25% by weight relative to the total weight of the cosmetic composition.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice or methylene blue.

The cosmetic composition according to the invention may also contain at least one material with a specific optical effect.

This effect is different from a simple conventional hue effect, i.e. a unified and stabilized effect as produced by standard dyestuffs, for instance monochromatic pigments.

For the purposes of the invention, the term “stabilized” means lacking the effect of variability of the colour with the angle of observation or in response to a temperature change.

For example, this material may be selected from the group constituted of particles with a metallic glint, goniochromatic colouring agents, diffracting pigments, thermochromic agents, optical brighteners, and also fibres, such as interference fibres. Needless to say, these various materials may be combined so as to afford the simultaneous manifestation of two effects, or even of a novel effect in accordance with the invention.

The particles with a metallic glint that may be used in the invention are for instance selected from the group constituted of:
particles of at least one metal and/or of at least one metal derivative,
particles comprising a monomaterial or multimaterial organic or mineral substrate, at least partially coated with at least one layer with a metallic glint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are preferred metals.

The term “metal derivatives” is intended to denote compounds derived from metals, such as oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe® 700 from the company Eckart, silica-coated aluminium particles sold under the name Visionaire Bright Silver® from the company Eckart, and metal alloy particles, for instance the silica-coated bronze (alloy of copper and zinc) powders sold under the name Visionaire Bright Natural Gold® from the company Eckart.

They may also be particles comprising a glass substrate, for instance those sold by the company Nippon Sheet Glass under the name Microglass Metashine®.

The goniochromatic colouring agent may be chosen, for example, from interference multilayer structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair® by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, structures being sold under the name Sicopearl® by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of Infinite Colors® pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter®.

Examples of liquid-crystal goniochromatic particles that may be used include those sold by the company Chenix and also the product sold under the name Helicone® HC by the company Wacker.

According to one embodiment, a composition according to the invention may comprise at least one dyestuff chosen, for example, from organic dyestuffs and inorganic dyestuffs, such as pigments and nacres, and materials with a specific optical effect, and mixtures thereof.

According to one embodiment, a composition according to the invention may comprise not more than 30% by weight of dyestuff relative to the total weight of the composition.

### Fillers

The cosmetic compositions in accordance with the invention may also comprise at least one filler, of organic or mineral nature, which for example makes it possible to give them improved stability with regard to exudation.

The term “filler” should be understood as meaning colourless or white solid particles of any form, which are in an insoluble form and dispersed in the medium of the composition. Being of mineral or organic nature they make it possible to impart body or rigidity to the composition, and/or softness, a matt effect and uniformity to the makeup.

The fillers used in the compositions according to the present invention may be of lamellar, globular or spherical form, fibres or in any other form intermediate between these defined forms.

The fillers according to the invention may or may not be surface-coated, and for instance they may be surface-treated with silicones, amino acids, fluoro derivatives or any other substance that promotes the dispersion and compatibility of the filler in the composition.

For the purposes of the present invention, the terms “mineral fillers” and “inorganic fillers” are used interchangeably.

Among the mineral fillers that may be used in the compositions according to the invention, mention may be made of talc, mica, silica, trimethyl siloxysilicate, kaolin, bentone, precipitated calcium carbonate, magnesium carbonate, magnesium hydrogen-carbonate, hydroxyapatite, boron nitride, hollow silica microspheres (Silica Beads® from Maprecos), glass or ceramic microcapsules, silica-based fillers, for instance Aerosil® 200 or Aerosil® 300; Sunsphere® L-31 and Sunsphere® H-31 sold by Asahi Glass; Chemicelen® sold by Asahi Chemical; composites of silica and of titanium dioxide, for instance the TSG series sold by Nippon Sheet Glass, and mixtures thereof.

Among the organic fillers that may be used in the compositions according to the invention, mention may be made of polyamide powders (Nylon® Orgasol from Atochem), poly-β-alanine powder and polyethylene powder, polytetrafluoroethylene powders (Teflon®), N-lauroyl-L-lysine, starch, tetrafluoroethylene polymer powders, hollow polymer microspheres such as Expancel® (Nobel Industrie), precipitated calcium carbonate, magnesium carbonate, magnesium hydrogen carbonate, metal soaps derived from organic carboxylic acids containing from 8 to 22 carbon atoms and for example from 12 to 18 carbon atoms, for example zinc stearate, magnesium stearate or lithium stearate, zinc laurate or magnesium myristate, Polypore® L 200 (Chemdal Corporation), silicone resin microbeads (for example Tospearl® from Toshiba), polyurethane powders, for instance powders of crosslinked polyurethane comprising a copolymer, the said copolymer comprising trimethylol hexyllactone. For example, it may be a polymer of hexamethylene diisocyanate/trimethylol hexyllactone. Such particles are for instance commercially available, for example, under the name Plastic Powder D-400® or Plastic Powder D-800® from the company Toshiki, and mixtures thereof.

A filler may be present in a cosmetic composition in accordance with the invention in a proportion of from 0.5% to 40% by weight and for example from 5% to 30% by weight of filler relative to the total weight of the composition.

A filler that is suitable for the invention may be, for example, a filler whose mean particle size is less than 100 μm and for instance between 1 and 50 μm, for example between 4 and 20 μm.

### Additives

A cosmetic composition according to the invention may, furthermore, also comprise any additive usually used in the field under consideration, selected from the group constituted of film-forming agents and, where appropriate, auxiliary film-forming agents, gums, semi-crystalline polymers, gelling agents, antioxidants, essential oils, preserving agents, fragrances, neutralizers, moisturizers, antiseptics, vitamins such as vitamin B3 or E and derivatives thereof, and anti-UV agents, and mixtures thereof.

### Colour Staying Power of the Composition

The term “colour staying power” is intended to denote the property of a cosmetic composition according to the invention of transferring to a minimum extent onto objects with which it may come into contact, and the property of withstanding interaction with liquids, for instance tears or sweat, or contact with food during a meal, for instance in the case, for example, of lipstick, and the property of not migrating from the initial line of the makeup, for example in the case of lipsticks, into the wrinkles and fine lines around the lips.

A film of cosmetic composition applied to the skin, mucous membranes and/or the lips may generally be impaired during contact with liquids, such as water or drinks consumed, for example, during a meal, or alternatively oils, for instance food oils, or else sebum or saliva.

Thus, the colour staying power of a cosmetic composition may be characterized by at least one of the following parameters: the colour resistance to oil, the transfer resistance and the colour resistance to water.

Measurement of the colour resistance to oil may be an adequate parameter for characterizing a composition according to the invention, and may be performed as follows.

The measurements are taken, for example, on the inner face of the forearm, washed and dried naturally at room temperature for 5 minutes. The cosmetic composition to be tested, for example a lipstick, is applied to five areas of the inner face of the forearm. The area of skin on which the measurements are taken should be at least greater than 1 cm². In general, the measurements are taken on circular areas equal to about 3 cm in diameter.

It is necessary for about the same amount of cosmetic composition to be applied to each of the three areas. This may be checked by measuring the weight of the first and the second cosmetic composition, after each of the applications, or by preparing beforehand equivalent amounts of sample to be tested. In general, for a surface area of 1 cm², an amount equal to about 2 mg is necessary (if the surface area has a diameter of 3 cm, then an amount of about 14 mg is required).

After applying the cosmetic composition, the colour, L₁*a₁*b₁*(C_{I}), is measured at each of the three areas, and the mean value obtained corresponds to the initial colour of the product. The colour measurement may be performed using a Minolta colorimeter of the CR300, CM500, CM1000 or CM2000 series. The Minolta calorimeter of the CR300 series is used in particular.

The test of colour resistance to oil is performed by applying to the test areas about 14 mg/cm² of oil of food type onto each area of the forearm (rapeseed oil, soybean oil or sunflower oil) followed by manual massaging for a few seconds, for example for 2 to 5 seconds and in instance for 2 seconds. A thickness of a commercial white paper handkerchief such as a Kleenex tissue is then applied to the area for about 5 seconds at a force of about 100 g/f, this force possibly being applied using a DPZ-5N digital pressure dynamometer from the manufacturer Imada Co. Ltd.

The value of the colour resistance to oil, H, is equal to the difference between the mean colour L₂*a₂*b₂*(Cₚ) of the composition remaining on the forearm after massaging with oil and application of the handkerchief, and the mean colour L₁*a₁*b₁*(C_{I}) initially measured,
ΔE (H)=\sqrt{(L₂*−L₁*)²+(a₂*−a₁*)²+(b₂*−b₁*)²}

The test of colour resistance to oil is a test that for example makes it possible to evaluate the remanence of a cosmetic composition such as a lipstick during a meal.

For example, the colour resistance to oil of a deposit of a composition according to the invention, once spread onto a support, may be less than or equal to about 20, less than or equal to about 15, less than or equal to about 10, or less than or equal to about 8.

### Gloss and Gloss Staying Power

The term “gloss” is intended to denote the gloss of a composition as may be conventionally measured using a glossmeter, via methods known to those skilled in the art.

For example, the gloss may be measured using a test performed in vitro using a Muti-Gloss 268 glossmeter (Minolta, Tokyo, Japan).

Such a test may be performed on a sample of Bio-Skin synthetic skin (of the type Fat-Skin Beaulax, Tokyo, Japan) 3 cm×4.5 cm in size prepared by applying a first coat of cosmetic composition, for example a lipstick, in the direction of the length of the support, in the form of continuous strips, so as to cover an area of 2.5 cm×4 cm. A second coat of the composition is applied over the first coat in a similar manner to the first, offset widthwise by about half the width of a strip in order to obtain a uniformly covered surface.

After applying the two coats of compositions, the gloss is measured with a glossmeter by measuring the reflectance at an angle of about 60° taken relative to the perpendicular to the surface. The gloss is for example measured 10 minutes after applying the cosmetic composition.

The measurement is repeated, for example at least five times, and a mean is determined of the values remaining after removing the lowest value and the highest value.

A composition according to the invention may have a gloss at least greater than or equal to 7, greater than or equal to 8 and greater than or equal to 9.

The term “gloss staying power” is intended to denote the remanence of the gloss of a composition as may be measured using a glossmeter via methods known to those skilled in the art.

For example, the protocol as defined above may be followed by applying a fabric to the sample of synthetic skin comprising the two coats of cosmetic compositions. Such a fabric may be, for example, a Kleenex tissue.

The fabric may be applied using a press, for example of mass 1.5 kg, for a defined time, for example 2 seconds, in a controlled manner with a rheometer (Exponet 32 texture).

Next, the gloss of the sample is measured as defined above, and the value obtained is indicative of the remanence.

A composition according to the invention may have a gloss staying power of greater than or equal to about 4, for example greater than or equal to about 5, for example greater than about 6.

According to one embodiment, a subject of the present invention is the use of at least one ester of dimerdilinoleic acid and of polyol(s) or an ester thereof, the viscosity of which, measured at about 25° C., is greater than or equal to about 20 000 mPa.s, in combination with at least one volatile oil and at least one film-forming agent, for the preparation of a cosmetic composition for caring for and/or making up the skin and/or the lips, which has improved gloss staying power, or even also improved colour staying power.

For instance, the ester, the film-forming agent and the volatile oil may be, for example, as defined above.

A composition in accordance with the invention may be prepared in the usual manner by a person skilled in the art. It may thus be in cast form, for example in the form of a stick or wand, or in the form of a soft paste in a heating bag or in the form of dishes that may be used by direct contact or with a sponge.

For example, it may constitute a cast foundation, a cast makeup rouge or eyeshadow, which is for instance coloured, a lipstick, a lip gloss or a concealer product.

A composition according to the invention may be obtained by heating the various constituents to the melting point of the highest-melting waxes, followed by pouring the molten mixture into a mould (crucible or finger stall). It may also be obtained by extrusion, as described in patent application EP-A-0 667 146.

The present invention also relates to a cosmetic method for making up and/or caring for the skin and/or the lips, comprising at least one step that consists in applying a composition in accordance with the invention to at least part of a support.

The present invention further relates to a cosmetic method for the preparation of a cosmetic composition for caring for and/or making up the skin and/or the lips, which has improved colour staying power comprising at least a step that consists in combining at least one ester of dimerdilicoleic acid and of polyol(s), or an ester thereof, the viscosity of which, measured at 25° C., is greater than or equal to 20 000 mPa.s with at least one silicone surfactant. For example, said ester and said silicone surfactant may be as defined above.

According to another of its aspects, a subject of the present invention is a synthetic support comprising a composition in accordance with the invention on at least part of its surface.

A cosmetic composition according to the invention may for example be in the form of a lip makeup and/or care composition, such as a lipstick, a lip balm or a gloss.

For the purposes of the present invention, and unless otherwise indicated, the word “one” should be understood as meaning “at least one”.

The examples of compositions below are given as illustrations and with no limiting nature on the invention.

### EXAMPLE 1

Lipstick

| Compound | % mass |
|---|---|
| Polyglyceryl-3 polymethylsiloxyethyl | 10.00 |
| dimethicone (KF-6104 from Shin-Etsu) | |
| Isononyl isononanoate | 6.60 |
| Polybutylene | 5.00 |
| Neopentyl glycol dicaprate | 25.00 |
| Hydrogenated polyisobutene | 5.00 |
| BHT | 0.05 |
| Silica dimethylsilylate | 2.00 |
| Polyglyceryl-2 isostearate | 10.00 |
| dimerdilinoleate copolymer | |
| (Hailuscent ISDA (HAI)) | |
| Bis-behenyl/isostearyl/phytosteryl- | 5.60 |
| dimerdilinoleyl dimerdilinoleate | |
| (Plandool-G (NFC)) | |
| Polyethylene (M = 500) | 6.60 |
| Candelilla wax | 2.00 |
| Polyglyceryl-2 triisostearate | 10.00 |
| Isononyl isononanoate | 3.00 |
| Titanium dioxide | 0.20 |
| D&C Red 7 W 012 C | 0.45 |
| Iron oxides | 0.95 |
| Yellow 5 Lake | 0.85 |
| Blue 1 Lake | 0.20 |
| Ethylene glycol dimethacrylate/lauryl | 1.00 |
| methacrylate copolymer | |
| N-Lauroyl-L-lysine | 1.00 |
| Flamingo Orange 320 C | 2.80 |
| Timiron Super Silk | 0.50 |
| MP 1005 | |
| Timiron Super Red | 1.00 |
| Simethicone | 0.20 |
| TOTAL | 100 |

### Procedure

An oily phase is prepared by mixing, while heating (about 95° C.), the oils, the silicone surfactant and the polyglyceryl-2 isostearate dimerdilinoleate copolymer (Hailuscent ISDA).

The oily phase thus prepared is stirred at about 95° C. and the fillers are added to the mixture.

The waxes, the pigments in the form of a pigmentary paste, prepared by mixing the dyestuffs with the polyglyceryl-2 triisostearate, the isononyl isononanoate, the simethicone and the bis-behenyl/isostearyl/phytosteryl dimerdilinoleyl dimerdilinoleate (Plandool-G) are then added to the mixture.

The mixture thus obtained is then poured into a lipstick mould and left to cool until a solid composition is obtained.

The gloss staying power of the composition is measured as described previously, and has a value of 6.3.

1. An anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa.s, and at least one silicone surfactant.

2. The composition according to claim 1, wherein the said ester has a molecular weight ranging from about 2000 to about 25 000 g/mol.

3. The composition according to claim 1, wherein the polyol is a diol.

4. The composition according to claim 2, wherein the ester comprises an alternating sequence of dimerdilinoleate residue(s) and of residue(s) related to the said diol(s).

5. The composition according to claim 3, wherein the diol is selected from the group constituted of a fatty alcohol dimer, a monoglycerol or polyglycerol, a C₂-C₄ monoalkylene or polyalkylene glycol, 1,4-butanediol and pentaerythritol.

6. The composition according to claim 5, wherein the fatty alcohol dimer is the product of hydrogenation of a fatty acid dimer obtained by dimerization of a C₈-C₃₄ unsaturated fatty acid.

7. The composition according to claim 4, wherein each of the two ends of the said sequence respectively bears a unit OR′ and OR″, with R′ and R″ representing, independently of each other, a hydrogen atom or OR′ and OR″ representing, independently of each other, a C₂-C₃₆ hydrocarbon-based monoalcohol residue.

8. The composition according to claim 7, wherein R′ and R″ both represent a hydrogen atom.

9. The composition according to claim 7, wherein OR′ and OR″ both represent an identical or different hydrocarbon-based monoalcohol residue.

10. The composition according to claim 1, comprising an ester of general formula (I) below:
R₃—OCO—R₁(—COO—R₂—OCO—R₁)ₙ—COO—R₃ (I)
wherein:
COR₁CO represents a dimerdilinoleate residue,
OR₂O represents a fatty alcohol dimer residue,
OR₃ represents a hydrocarbon-based monoalcohol residue, and
n is an integer ranging from 1 to 15.

11. The composition according to claim 10, wherein OR₂O represents a dimerdilinoleyl residue.

12. The composition according to claim 10, wherein OR₃ represents a hydrocarbon-based monoalcohol residue selected from the group constituted of behenyl, isostearyl and phytosteryl residues, and mixtures thereof.

13. (canceled)

14. The composition according to claim 1, comprising an ester of general formula (II) below: 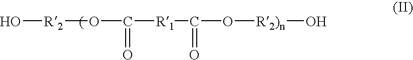
wherein:
n is an integer ranging from 1 to 15,
COR′₁CO represents a dimerdilinoleate residue,
OR′₂O represents a diglyceryl residue of general formula (III) below: 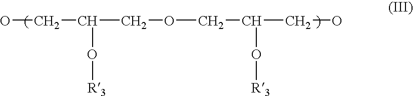
wherein:
R′₃ represents H or OR′₃ represents a fatty acid residue.

15. The composition according to claim 14, wherein the fatty acid residue featured by OR′₃ is an isostearyl residue.

16. The composition according to claim 1, wherein the said ester is selected from the group constituted of the esters having the following INCI nomenclature: polyglyceryl-2 isostearate/dimerdilinoleate copolymer, bisbehenyl/isostearyl/phytosteryl dimerdilinoleyl dimerdilinoleate, and mixtures thereof.

17. The composition according to claim 1, wherein it comprises from 5% to 90% by weight of the said ester relative to the total weight of the composition.

18. The composition according to claim 1, wherein the silicone surfactant is selected from the group constituted of a polyhydroxylated silicone surfactant and an emulsifying silicone resin or elastomer, and mixtures thereof.

19. The composition according to claim 1, wherein the polyhydroxylated silicone surfactant is of general formula (IV):
R¹ₐR²_{b}R³_{c}SiO_{(4-a-b-c)/2} (IV)
wherein:
a) a, b and c are such that a ranges from 1 to 2.5; and b and c, independently of each other, range from 0.001 to 1.5,
b) R¹, which may be identical or different, is selected from the group constituted of:
C₁ to C₃₀ alkyl radicals, where appropriate substituted with one or more fluorine atoms and amino and/or carboxyl groups,
aryl and aralkyl radicals, and
the radicals of general formula (V):
—C_{d}H_{2d}—O—(C₂H₄O)ₑ(C₃H₆O)_{f}R⁴ (V)
wherein:
R⁴ is a C₁ to C₃₀ hydrocarbon-based radical or a radical R⁵—(Co)— with R⁵ being a C₁ to C₃₀ hydrocarbon-based radical, and
d, e and f are integers such that d ranges from 0 to 15, and e and f, independently of each other, range from 0 to 50, and
combinations thereof,
c) R² is a radical represented by the general formula (VI):
-Q-O—X (VI)
wherein:
Q is a divalent C₂ to C₂₀ hydrocarbon-based radical optionally including at least one ether bond and/or at least one ester bond, and
X is a polyhydroxylated hydrocarbon-based radical,
d) R³ is an organosiloxane group of general formula (VII): 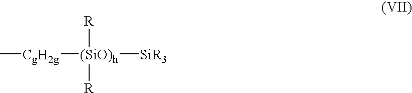
with:
each of the radicals R representing, independently of each other, a radical selected from the group constituted of C₁ to C₃₀ alkyl radicals, where appropriate substituted with one or more fluorine atoms, and aryl and aralkyl radicals,
g and h are integers such that g ranges from 1 to 5 and h ranges from 0 to 500.

20. The composition according to claim 19, wherein:
a ranges from 1 to 1.4, and b and c, independently of each other, range from 0.02 to 0.04, and
R¹ is a C₁ to C₁₀ alkyl radical,
R² is represented by the formula (VIA):
—C₃H₆O[CH₂CH(OH)CH₂O]ₙH (VIA)
wherein n ranges from 1 to 5, and
R³ is represented by the formula (VIIA):
—C₂H₄(CH₃)₂SiO[(CH₃)₂SiO]ₘSi(CH₃)₃ (VIIA)
wherein m ranges from 3 to 9.

21. The composition according to claim 20, wherein R¹ is a methyl radical.

22. The composition according to claim 18, wherein the polyhydroxylated silicone surfactant is selected from the group constituted of polyglyceryl-3 polymethylsiloxyethyl dimethicone, laurylpolyglyceryl-3 polymethylsiloxyethyl dimethicone and polyglyceryl-3 disiloxane dimethicone, and mixtures thereof.

23. The cosmetic composition according to claim 18, wherein the emulsifying silicone elastomer is selected from the group constituted of polyoxyalkylenated silicone elastomers, and mixtures thereof.

24. The composition according to claim 1, further comprising at least one liquid fatty phase selected from the group constituted of volatile oils and non-volatile oils, and mixtures thereof.

25. The composition according to claim 24, wherein the said volatile oil is selected from the group constituted of:
hydrocarbon-based oils containing from 8 to 16 carbon atoms,
linear or cyclic silicone oils having a viscosity ≦8 centistokes (8×10⁻⁶ m²/s),
volatile fluoro oils such as nonafluoromethoxybutane or perfluoromethyl-cyclopentane, and
mixtures thereof.

26. The composition according to claim 24, wherein the said non-volatile oil is selected from the group constituted of oils of animal origin, oils of plant origin and oils of synthetic or mineral origin, and mixtures thereof.

27. The composition according to claim 1, further comprising at least one solid fatty phase selected from the group constituted of waxes and pasty fatty substances, and mixtures thereof.

28. The composition according to claim 1, further comprising at least one dyestuff.

29. The composition according to claim 28, wherein the dyestuff is selected from the group constituted of organic dyestuffs and inorganic dyestuffs.

30. The composition according to claim 1, wherein it is in the form of a suspension, a dispersion, a solution, a gel, an emulsion, or a multiple emulsion (W/O/W or polyol/O/W or O/W/O) or in the form of a cream, a stick, a paste, a foam, a vesicular dispersion, a two-phase or multi-phase lotion or a powder.

31. The composition according to claim 1, said composition being a lip makeup and/or care composition.

32. A cosmetic composition for caring for and/or making up the skin and/or the lips, comprising at least one ester of general formula (I) below:
R₃—OCO—R₁(—COO—R₂—OCO—R₁)ₙ—COO—R₃ (I)
wherein which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15,
and at least one silicone surfactant.

33. The composition according to claim 32, wherein in which OR₂O represents a dimerdilinoleyl residue.

34. The Ccomposition according to claim 32, wherein in which OR₃ represents a hydrocarbon-based monoalcohol residue selected from the group constituted of behenyl, isostearyl and phytosteryl residues, and mixtures thereof.

35. The composition according to claim 32, said composition being anhydrous.

36. A cosmetic composition for caring for and/or making up the skin and/or the lips, comprising at least one ester of general formula (II) below: 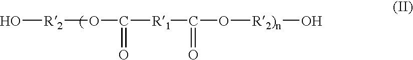
wherein:
n is an integer ranging from 1 to 15,
COR′₁CO represents a dimerdilinoleate residue,
OR′₂O represents a diglyceryl residue of general formula (m) below: 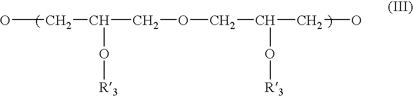
wherein:
R′₃ represents H or OR′₃ represents a fatty acid residue, and comprising at least one silicone surfactant.

37. The composition according to claim 36, wherein the fatty acid residue featured by OR′₃ is an isostearyl residue.

38. The composition according to claim 36, said composition being anhydrous.

39. A cosmetic method for the preparation of a cosmetic composition for caring for and/or making up the skin and/or the lips, which has improved colour staying power, comprising at least a step that consists in combining at least one ester of dimerdilinoleic acid and of polyol(s), or an ester thereof, the viscosity of which, measured at 25° C., is greater than or equal to 20 000 mPa.s, with at least one silicone surfactant.

40. A cosmetic method for the preparation of a cosmetic composition for caring for and/or making up the skin and/or the lips, which has improved colour staying power, comprising at least a step that consists in combining at least one ester of dimerdilinoleic acid and of polyol(s), or an ester thereof, the viscosity of which, measured at 25° C., is greater than or equal to 20 000 mPa.s, with at least one silicone surfactant, wherein the said ester is as defined according to claim 1.

41. A cosmetic method for the preparation of a cosmetic composition for caring for and/or making up the skin and/or the lips, which has improved colour staying power, comprising at least a step that consists in combining at least one ester of dimerdilinoleic acid and of polyol(s), or an ester thereof, the viscosity of which, measured at 25° C., is greater than or equal to 20 000 mPa.s, with at least one silicone surfactant, wherein the silicone surfactant is as defined according to claim 18.

42. The cosmetic method for making up and/or caring for the skin and/or the lips, comprising at least one step that consists in applying to at least a portion of the said support a composition as defined according to claim 1.
